(19) United States
(12) Patent Application Publication
(10) Pub. No.: US 2026/0207247 A1
(43) Pub. Date: Jul. 23, 2026

(54) A Medical Device for Forming a Fistula

(71) Applicant: CLEARSTREAM TECHNOLOGIES LIMITED, Enniscorthy, County Wexford (IE)

(72) Inventors: Sean Wall, Enniscorthy, County Wexford (IE); Michael Whelan, Enniscorthy, County Wexford (IE); John O'Shea, Enniscorthy, County Wexford (IE)

(21) Appl. No.: 19/140,822

(22) PCT Filed: Dec. 20, 2022
(86) PCT No.: PCT/EP2022/086936

§ 371 Date: Jun. 18, 2025

(51) Int. Cl. A61B 18/14; A61B 18/00; A61B 18/08

(52) U.S. Cl. CPC A61B 18/14; A61B 18/082; A61B 2018/00184; A61B 2018/00601; A61B 2018/142

(57) ABSTRACT

A medical device for forming a fistula comprises a body portion having a longitudinal axis; a head portion positioned distally of the body portion and configured to be longitudinally moveable relative to the body portion to capture tissue in which the fistula is to be formed between the head portion and the body portion; a first electrical cutting element for cutting the tissue captured between the head portion and body portion; and a cutting edge for cutting the tissue captured between the head portion and body portion.

### TECHNICAL FIELD

The present disclosure relates to a medical device for forming a fistula between two vessels.

### BACKGROUND

A fistula denotes a passageway formed between two internal organs. Forming a fistula between two blood vessels can have one or more beneficial functions. For example, the formation of a fistula between an artery and a vein may provide access to the vasculature for haemodialysis patients. Specifically, forming a fistula between an artery and a vein allows blood to flow quickly between the vessels while bypassing the capillaries. Needles, catheters, or other cannulas may then be inserted into the blood vessels near the fistula to draw blood from the circulatory system, pass it through a dialysis machine, and return it to the body. The quickened flow provided by the fistula may provide for effective haemodialysis.

The formation of a fistula may also be helpful in the treatment of peripheral arterial disease (PAD), for example. PAD can result from the occlusion of arteries in the legs and lower extremities such as the feet. Typically, such occlusion is brought about by atherosclerosis, whereby calcified plaque deposited on the walls of an arterial vessel causes narrowing and blockage of the vessel lumen. Treatment for PAD in severe cases may include deep vein arterialisation (DVA), whereby a fistula may be formed between a diseased artery and a nearby vein to route the blood flow from the diseased artery to the nearby vein in order to supply the extremity with blood. Another possible treatment for PAD is an endovascular bypass procedure, whereby two fistulas are formed either side of a blockage in a diseased artery. The blood flow is routed out of the artery and back into the artery by a conduit that circumvents the blockage. That conduit may, for example, be a stent graft placed within an adjacent vein.

In view of the above, there exists a need in the art for a medical device for which can effectively and safely form a fistula.

### SUMMARY

According to the present disclosure, there is provided a medical device for forming a fistula. The medical device comprises a body portion having a longitudinal axis; a head portion positioned distally of the body portion and configured to be longitudinally moveable relative to the body portion to capture tissue in which the fistula is to be formed between the head portion and the body portion; a first electrical cutting element for cutting the tissue captured between the head portion and body portion; and a cutting edge for cutting the tissue captured between the head portion and body portion.

In some embodiments, this may result in a medical device which can effectively and safely form a fistula.

The medical device may have a closed configuration where the head portion and the body portion are in contact with each other, and an open configuration where the head portion and the body portion are longitudinally spaced apart from each other.

The first electrical cutting element and the cutting edge may be disposed opposite each other on the body portion and head portion. In the closed configuration, the first electrical cutting element and the cutting edge may be in contact with each other.

In some embodiments, this may result in more effective formation of the fistula due to the first electrical cutting element and the cutting edge cutting at the same position.

The first electrical cutting element and the cutting edge may be disposed together on the head portion or the body portion.

The first electrical cutting element and the cutting edge may be disposed at the same position on the head portion or the body portion.

In some embodiments, this may result in more effective formation of the fistula due to the first electrical cutting element and the cutting edge cutting at the same position.

The first electrical cutting element and the cutting edge may be positioned adjacent each other on the head portion or the body portion.

The first electrical cutting element may be circumferentially disposed on the head portion or the body portion.

In some embodiments, this may result in more effective formation of the fistula as this allows the first electrical cutting element to cut the entire circumference of the fistula.

The first electrical cutting element may be in the form of a circumferential band disposed on the head portion or the body portion.

The cutting edge may be a circumferential cutting edge.

In some embodiments, this may result in more effective formation of the fistula as this allows the cutting edge to cut the entire circumference of the fistula.

The first electrical cutting element may be a resistive electrical heating element.

In some embodiments, this may provide for an effective cutting action by the first electrical cutting element.

The first electrical cutting element may be an RF electrode.

In some embodiments, this may provide for an effective cutting action by the first electrical cutting element.

The body portion may comprise a protruding distal end portion. The head portion may comprise a complementary recessed proximal end portion for engaging the distal end portion of the body portion.

In some embodiments, this may allow for effective capturing of the tissue in which the fistula is to be formed between the head portion and the body portion.

The recessed proximal end portion of the head portion may define a cavity with a proximal opening.

In some embodiments, this may allow the tissue in which the fistula is to be formed to be captured in the cavity to allow a safe formation of the fistula.

The cutting edge may be positioned on a proximal leading edge of the recessed proximal end portion.

The first electrical cutting element may be positioned on the protruding distal end portion.

The protruding distal end portion may be substantially cone shaped.

In some embodiments, this may allow for effective capturing of the tissue in which the fistula is to be formed between the head portion and the body portion.

The first electrical cutting element may be positioned at a base of the cone-shaped protruding distal end portion.

The body portion may comprise a recessed distal end portion. The head portion may comprise a complementary protruding proximal end portion for engaging the distal end portion of the body portion.

In some embodiments, this may allow for effective capturing of the tissue in which the fistula is to be formed between the head portion and the body portion.

The recessed distal end portion of the body portion may define a cavity with a distal opening.

In some embodiments, this may allow the tissue in which the fistula is to be formed to be captured in the cavity to allow a safe formation of the fistula.

The cutting edge may be positioned on a distal leading edge of the recessed distal end portion.

The first electrical cutting element may be positioned on the protruding proximal end portion.

The protruding proximal end portion may be substantially cone shaped.

In some embodiments, this may allow for effective capturing of the tissue in which the fistula is to be formed between the head portion and the body portion.

The first electrical cutting element may be positioned at a base of the cone-shaped protruding proximal end portion.

The medical device may further comprise a second electrical cutting element positioned opposite the first electrical cutting element on the body portion or the head portion.

In some embodiments, this may result in more effective formation of the fistula, as the first and second electrical cutting element can

The second electrical cutting element may be a resistive electrical heating element or an RF electrode.

In some embodiments, this may provide for an effective cutting action by the second electrical cutting element.

The second electrical cutting element may be circumferentially disposed on the body portion or the head portion.

The second electrical cutting element may be in the form of a circumferential band disposed on the body portion or the head portion.

The first electrical cutting element may form a raised ridge.

In some embodiments, this may exert more pressure on the tissue to be cut, thereby resulting in more effective formation of the fistula.

The second electrical cutting element may form a raised ridge.

In some embodiments, this may exert more pressure on the tissue to be cut, thereby resulting in more effective formation of the fistula.

The body portion may be an elongate body portion.

In some embodiments, this may allow the body portion to more easily enter a blood vessel where a fistula is to be formed.

The head portion may have a tapered shape.

In some embodiments, this may allow the head portion to more easily advance through a blood vessel and pass through an opening in a blood vessel.

The head portion may be substantially conical shaped.

In some embodiments, this may allow the head portion to more easily advance through a blood vessel and pass through an opening in a blood vessel.

The medical device may further comprise an inner shaft connected to the head portion and longitudinally moveable within the body portion.

In some embodiments, this may provide an effective and simple way to move the head portion longitudinally relative to the body portion.

The inner shaft may comprise a guidewire lumen for accommodating a guidewire.

In some embodiments, this may allow the medical device to be tracked over a guidewire.

The inner shaft may comprise one or more flushing ports for injecting a contrast agent.

In some embodiments, this may allow for an effective way to test the patency of the fistula after formation.

In the open configuration, the one or more flushing ports may be disposed between the body portion and the head portion.

In some embodiments, this may allow for an effective way to test the patency of the fistula after formation.

The inner shaft may comprise a flushing lumen connected to the one or more flushing ports.

In some embodiments, this may allow a contrast agent to be injected through the flushing ports.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1A shows a cross-sectional view of medical device for forming a fistula in an open configuration according to one or more embodiments of the present disclosure;

FIG. 1B shows an isometric view of the medical device for forming a fistula of FIG. 1A.

FIG. 2A shows a cross-sectional view of the medical device of FIG. 1A in a closed configuration;

FIG. 2B shows a cross-sectional view along line AA of FIG. 2A;

FIG. 3A shows a needle and guidewire being introduced into an artery A and vein V;

FIG. 3B shows a cross-sectional view of the medical device of FIG. 1A being introduced into the artery A and vein V;

FIG. 3C shows a cross-sectional view of the medical device of FIG. 1A in the open configuration;

FIG. 3D shows a cross-sectional view of the medical device of FIG. 1A in the process of forming a fistula between the artery A and vein V;

FIG. 3E shows a cross-sectional view of the medical device of FIG. 1A after the fistula has been formed;

FIG. 3F shows a cross-sectional view the medical device of FIG. 1A injecting a contrast agent into the fistula;

FIG. 3G shows a cross-sectional view of the medical device of FIG. 1A being removed from the artery A and vein V;

FIG. 4 shows a cross sectional view of an alternative embodiment of a medical device according to one or more embodiments of the present disclosure;

FIG. 5 shows a cross-sectional view of an alternative embodiment of a medical device according to one or more embodiments of the present disclosure;

FIG. 6 shows a cross-sectional view an alternative embodiment of a medical device according to one or more embodiments of the present disclosure;

FIG. 7 shows a cross-sectional view of an alternative embodiment of a medical device according to one or more embodiments of the present disclosure.

### DETAILED DESCRIPTION

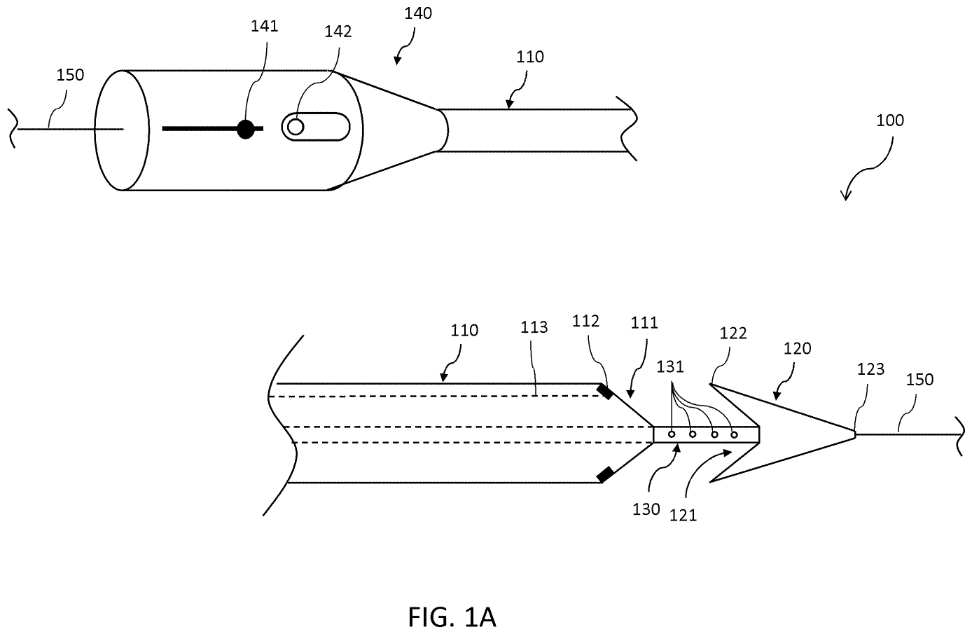

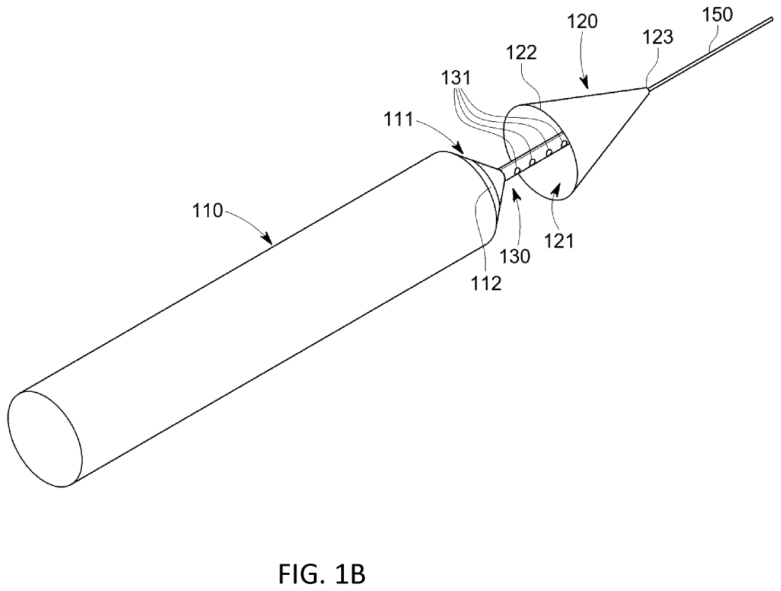

FIGS. 1A and 1B shows a medical device 100 for forming a fistula, such as a fistula between two blood vessels. FIG. 1A shows a proximal portion of the medical device with a handle 140 and a cross-sectional view of a distal portion of the medical device 100. FIG. 1B shows an isometric view of the distal portion of the medical device 100.

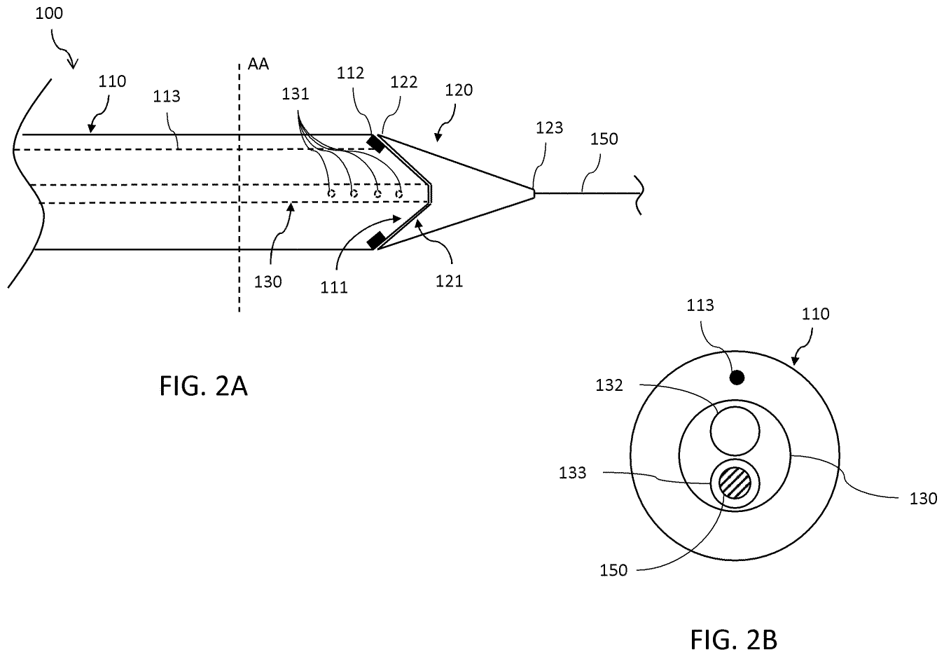

The medical device 100 comprises a body portion 110, which may be an elongate body portion 110 having a longitudinal axis, and a head portion 120 which is longitudinally moveable relative to the body portion 110. The head portion 120 may be moveable between a closed configuration (see FIG. 2A) where the body portion 110 and head portion 120 are in contact with each other, and an open configuration, shown in FIGS. 1A and 1B, where the head portion 120 and body portion 110 are longitudinally spaced apart from each other. The head portion 120 may be fixed to an inner shaft 130 which may be longitudinally moveable relative to the body portion 110 in order to move the head portion 120 between the open and closed configuration. By moving the head portion from the open to the closed configuration, tissue in which a fistula is to be formed, such as the walls of a blood vessel, for example, may be captured between the head portion 120 and the body portion 110. The medical device 100 further comprises an electrical cutting element 112 and a cutting edge 122 which can cut the tissue capture between the head portion 120 and the body portion 110 in order to form the fistula.

The body portion 110 may be an elongate body portion 110 and may have a cylindrical shape, for example. The body portion 110 may further have a protruding distal end portion 111. As shown in FIGS. 1A and 1B, the protruding distal end portion 111 may have a substantially conical or frustoconical shape. The electrical cutting element 112 may extend circumferentially around the body portion 110 (as can be seen in FIG. 1B), for example as a circumferential band, and may be positioned at a base of the protruding distal end portion 112. The electrical cutting element 112 may be in the form of an electrical heating element or an RF electrode, for example. A conducting element 113, such as a wire, may connect the electrical cutting element 112 at its distal end to a source of electrical energy, such as a generator, at its proximal end. The conducting element 113 may extend along the length of the body portion 110.

The head portion 120 is positioned distally c the body portion 110 and may have a recessed proximal end portion 121. The recessed proximal end portion 121 may be in the form of a cavity and may be shaped complementary to the protruding distal end portion 111 to allow the protruding distal end portion 111 and the recessed proximal end portion 121 to engage with each other. For example, as shown in FIGS. 1A and 1B, the recessed proximal end portion 121 may be a substantially conical or frustoconical cavity. The cutting edge 122 may be positioned at a leading proximal edge of the head portion 120. The cutting edge 122 and the electrical cutting element 112 may be positioned opposite each other on the head portion 120 and the body portion 110 respectively. The cutting edge 122 may extend circumferentially around the head portion (as can be seen in FIG. 1B) and may be in the form of a sharp blade for cutting the tissue captured between the head portion 120 and the body portion 110. The head portion 120 may have a tapered outer surface which tapers from a proximal end having a larger diameter to a distal end 123 with a smaller diameter. The tapered outer surface of the head portion 120 may be substantially conical, as shown in FIGS. 1A and 1B.

The inner shaft 130 may be at least partly disposed within an inner lumen of the body portion 120 and may extend along the length of the body portion 110. The inner shaft 130 may be longitudinally moveable with respect to the body portion 119. A distal end of the inner shaft 130 may be fixed to the head portion 120 such that longitudinal movement of the inner shaft 130 results in corresponding longitudinal movement of the head portion 120. The inner shaft 133 may also comprise one or more flushing ports 131. The one or more flushing ports 131 may be positioned between the body portion 110 and the head portion 120 in the open configuration. The one or more flushing ports 131 may be connected to a flushing lumen 132 (see FIG. 2B) disposed within the inner shaft 130 to allow a contrast agent to be delivered through the flushing lumen 132 and out of the one or more flushing ports 131. FIGS. 1A and 1B show the inner shaft having four flushing ports 131, however, the inner shaft may have any number of suitable flushing ports 131. For example, the inner shaft 130 may have 1 to 20 flushing ports.

The medical device 100 may comprise a guidewire lumen 133 (see FIG. 2B) for accommodating a guidewire 150. The guidewire lumen 133 may be positioned within the inner shaft 130 and the head portion 120 and may extend along the length of the medical device 100 to an opening at the distal end 123 of the head portion 120. The guidewire 150 may move longitudinally within the guidewire lumen 133 and may exit the guidewire lumen 133 at the opening at the distal end 123 of the head portion to extend distally past the head portion 120.

The medical device 110 may further comprise a control handle 140 disposed at the proximal end of the body portion 110. The control handle may comprise a capture control mechanism, which may be a slider 141 as shown in FIG. 1A. The slider 141 may be connected to the inner shaft 130 and may allow the inner shaft 130 and thereby the head portion 120 to be moved longitudinally relative to the body portion 110. For example, distal movement of the slider 141 may move the head portion 120 distally relative to the body portion whilst proximal movement of the slider 141 may move the head portion 120 proximally relative to the body portion 110. The control handle 140 may further have a switch 142 for activating the electrical cutting element 112. By toggling the switch, electrical current may be allowed to flow from the electrical energy source through the conducting element 113 to the electrical cutting element 112. The electrical current may be a radiofrequency (RF) current if the electrical cutting element 112 is an RF electrode, or may be a lower frequency AC current or DC current if the electrical cutting element 112 is an electrical heating element. The electrical energy source may be disposed within the control handle 140 or may be an external source connected to the control handle 140. The electrical energy source may be an electrosurgical unit (ESU), for example.

The electrical cutting element 112 may be made from a number of suitable materials, such as one or more refractory metals. For example, the electrical cutting element 122 may comprise tungsten, molybdenum, niobium, tantalum, rhenium, or combinations and alloys thereof. The head portion 120 and the distal end of the body portion 110 may be made from a non-conductive ceramic material which can withstand the heat generated by the electrical cutting element 112. The cutting edge 122 may be made from a number of suitable materials such as stainless steel or a nickel titanium alloy, for example.

FIG. 2A shows the medical device 100 in the closed configuration where the protruding distal end portion 111 of the body portion 110 and the recessed proximal end portion 121 of the head portion 120 are in contact with each other or positioned very close to each other. In order to move the head portion 120 from the open configuration to the closed configuration, the slider 141 may be moved proximally in order to move the inner shaft 130 and the head portion 120 proximally with respect to the body portion 110. As shown in FIG. 2A, in the closed configuration, the electrical cutting element 112 and the cutting edge 122 may be in contact with each other and the flushing ports 131 of the inner shaft 130 may be positioned within the body portion 110.

FIG. 2B shows a cross-sectional view of the body portion 110 along line AA. As shown in FIG. 2B, the body portion 110 may have a circular cross-section with conducting element 113 positioned within the body portion 113. The inner shaft 130 may also have a circular cross-section and may be positioned centrally within the body portion 110. The inner shaft 130 may have two separate lumens, one guidewire lumen 133 for accommodating the guidewire 150, and one flushing lumen 132 connected to the flushing ports 131 which may be used to inject a contrast agent into the fistula through the flushing ports 131.

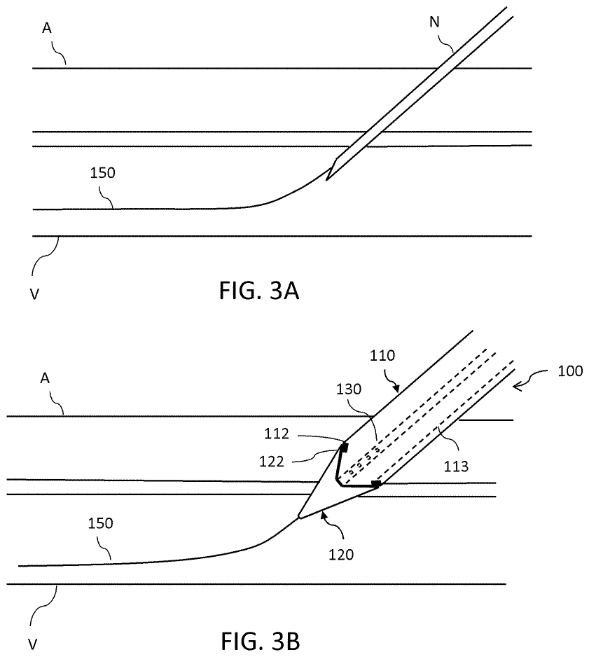

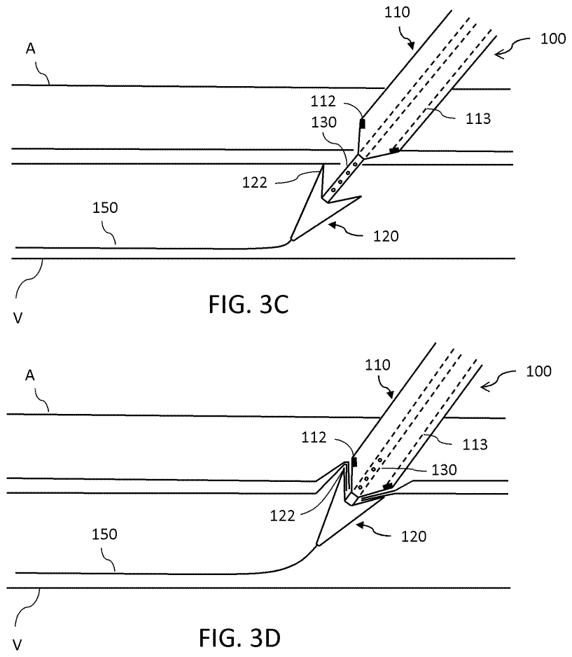

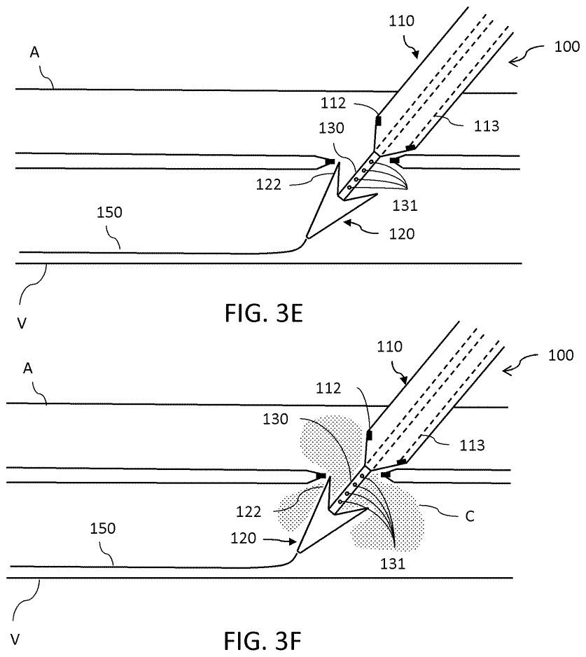

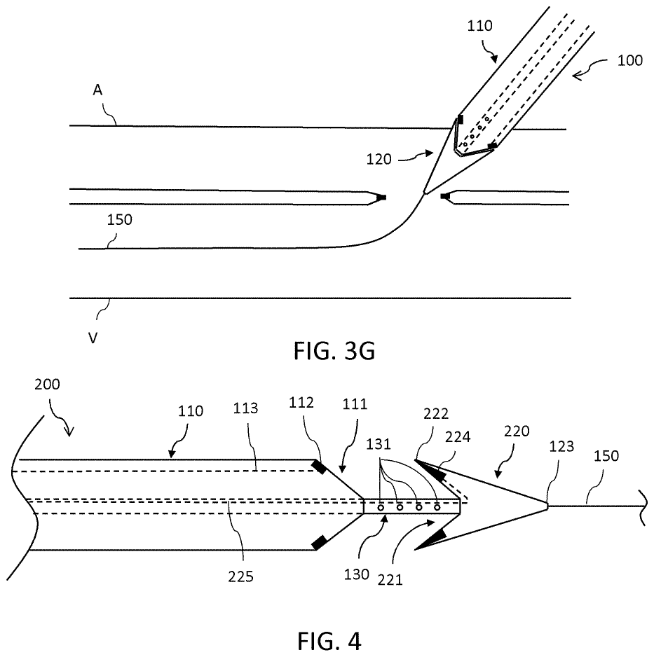

FIGS. 3A to 3E illustrate a method of using the medical device 100 in order to form a fistula between an artery A and a vein V and test the patency of the fistula.

Firstly, a suitable site for forming the fistula is identified and a needle N is inserted through the artery A and into the vein V. The needle N pierces the walls of the artery A and vein V and forms a small opening between the artery A and the vein V. As shown in FIG. 3A, the guidewire 150 is then introduced through a lumen of the needle N into the vein V.

As shown in FIG. 3B, the medical device 100 is then introduced over the guidewire 150 into the artery A and the vein V. During the introduction, the medical device 100 may be in the closed configuration. The tapered shape of the head portion 120 helps the head portion 120 to enter the vein V through the opening between the artery A and the vein V formed by the needle N.

Once the head portion 120 has passed through the opening and is positioned in the vein V, the medical device 100 may be moved from the closed position to the open position by moving the head portion 120 distally with respect to the body portion 110, as shown in FIG. 3C. The medical device 100 may be moved from the closed position to the open position, for example, by moving the slider 141 proximally. As shown in FIG. 3C, at this point the head portion 120 is fully positioned within the vein V whilst the body portion 110 is positioned within the artery A.

The medical device 100 is then moved from open configuration back to the closed configuration by moving the head portion 120 in a proximal direction relative to the body portion 110. As shown in FIG. 3D, this causes the tissue of the arterial wall and the venous wall around the opening to be captured between the head portion 120 and the body portion 110. The protruding distal end portion 111 of the body portion 110 pushes the tissue of the arterial wall and venous wall into the recessed proximal end portion of the head portion.

Once the tissue of the arterial wall and venous wall has been captured between the head portion 120 and the body portion 110, the electrical cutting element 112 may be activated, for example, by toggling the switch 142. This causes an electrical current to flow into the electrical cutting element 112. If the electrical cutting element 112 is an RF electrode, the electrical current may be an RF current. The RF current causes the RF electrode to heat up and generate a plasma which causes rapid dissociation and vaporization of the organic tissue of the arterial and venous walls captured between the head portion 120 and the body portion 110 to form a fistula. The RF electrode may also cause fusing of the arterial and venous walls. At the same time, the cutting edge 122 also mechanically cuts the tissue of the artery wall and the vein wall to form the fistula. A medical practitioner may pull the medical device 100 proximally at this point in order for the cutting edge to better engage and cut the tissue of the arterial and venous walls. This dual cutting action results in a very effective fistula formation. Furthermore, by having the electrical cutting element positioned at the base of the protruding distal end portion 111, the RF energy is directed mostly into the recessed proximal end portion 121 of the head portion 120, thereby minimising any damage to the arterial and venous walls outside of the fistula.

The method is substantially the same if the electrical cutting element 112 is an electrical heating element. In that case, the electrical current supplied to the electrical heating element may be lower frequency AC current or a DC current. The electrical heating element heats up and vaporizes the tissue of the arterial and venous walls. Furthermore, the electrical heating element will cause fusing of the arterial and venous walls. At the same time, the cutting edge 122 also mechanically cuts the tissue of the artery wall and the vein wall to form the fistula. This dual cutting action results in a very effective fistula formation.

The circumferential shape of the electrical cutting element 112 and the cutting edge 122 results in the effective formation of a circular fistula.

Once the fistula has been successfully formed, the medical device 100 may be moved again from the closed configuration to the open configuration, as shown in FIG. 3E. This may again be achieved moving the slider 141 distally, for example.

As shown in FIG. 3F, the patency of the fistula can then be tested by injecting a contrast agent C into the fistula through the flushing ports 131. In other words, a determination can be made as to how successful the fistula formation was and how well the fistula allows blood and other fluids to pass through. In order to do that, the medical device 100 is positioned in the open configuration so that the head portion 120 is positioned in the vein V whilst the body portion 110 is positioned in the artery A. The inner shaft 130 with the flushing ports 131 is positioned within the fistula. The contrast agent C, which can be viewed under fluoroscopy, for example, is then injected through the flushing lumen 132 and out of the flushing ports 131. A medical practitioner can then view how the contrast agent C moves through the fistula under fluoroscopy to determine whether the fistula formation has been successful. The contrast agent C may contain, for example, iodine or barium sulphate.

Once it has been determined that the fistula formation was successful, the medical device 100 can be moved to the closed configuration and removed from the patient, as shown in FIG. 3G.

The present medical device 100 therefore allows for safe and effective fistula formation through a dual electrical and mechanical cutting action and also provides for a simple and effective way to test the patency of the fistula after formation.

FIG. 4 shows an alternative embodiment of a medical device 200 for forming a fistula. The medical device 200 is similar to medical device 100 of FIGS. 1 to 3 and the same reference numerals are used to denote identical features which are not described in detail.

However, the medical device 200 differs from medical device 200 in that it comprises a second electrical cutting element 224 in addition to a first electrical cutting element 112. The second electrical cutting element 224 is positioned on the head portion 220, either at the same position or directly adjacent to the cutting edge 222, i.e. opposite the first electrical cutting element 112. The second electrical cutting element 224 may be in the form of an RF electrode or an electrical heating element and may be disposed circumferentially on the inside of the recessed proximal end portion 221 of the head portion 220, for example, in the form of a circumferential band. The second electrical cutting element 224 may be connected to a source of electrical energy with a second connector element 225 which is positioned within the inner shaft 130 and extends along the length of the inner shaft 130. The second electrical cutting element 224 may be made from the same materials as the first electrical cutting element 112.

The method of using the medical device 200 to form a fistula is substantially the same as that described above with respect to FIGS. 3A to 3G. However, during the fistula formation step (see FIG. 3D), both the first and second electrical cutting elements 112, 224 are activated, resulting in the tissue of the arterial and venous walls being cut from both sides, thus making the fistula formation process more effective.

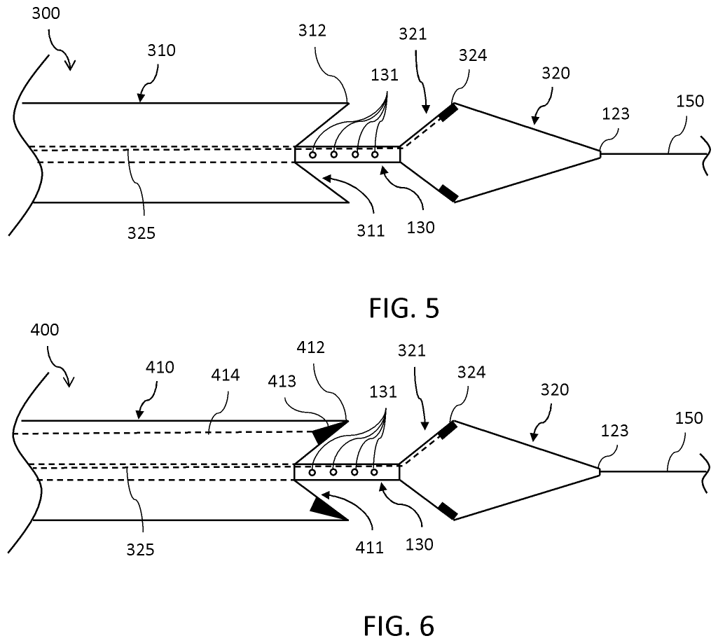

FIG. 5 shows another alternative embodiment of a medical device 300 for forming a fistula. The medical device 300 is similar to medical device 100 and the same reference numerals are used to denote identical features which are not described in detail.

The medical device 300 has a body portion 310 and a head portion 320 which is fixed to the inner shaft 130 and longitudinally moveable relative to the body portion 310. However, the body portion 310 comprises a recessed distal end portion 311 with the cutting edge 312 formed on a leading distal edge of the recessed distal end portion 311. The head portion 120 on the other hand comprises a protruding proximal end portion 321 which is shaped to engage the recessed distal end portion 311. As shown in FIG. 5, the protruding proximal end portion 321 may be substantially conical or frustoconical and the recessed distal end portion 311 may be a corresponding substantially conical or frustoconical cavity. The electrical cutting element 324 is disposed on the head portion 320 and specifically at a base of the protruding proximal end portion 321. The electrical cutting element 324 may be circumferential electrical cutting element which extends as a circumferential band around the head portion 320. A conducting element 325 may extend along inner shaft 130 to connect the electrical cutting element 324 to an electrical energy source. The electrical cutting element 324 may also be an RF electrode or an electrical heating element and may extend circumferentially around the head portion 120.

The method of using the medical device 300 to form a fistula is substantially the same as for medical device 100 described with reference to FIG. 3A to G above. The medical device 300 still provides for a dual cutting action with the cutting edge 312 and the electrical cutting element 324 forming the fistula simultaneously. The only difference is that the cutting edge 312 is formed on the body portion 310 whilst the electrical cutting element 324 is positioned on the head portion 320, rather than the other way around. During the fistula formation process, a medical practitioner may therefore push the medical device 300 distally during the fistula formation process (see FIG. 3D) to allow the cutting edge 312 to better engage the tissue of the arterial and venous walls.

FIG. 6 shows another alternative embodiment of a medical device 400 for forming a fistula. The medical device 200 is similar to medical devices 100 and 300 and the same reference numerals are used to denote identical features which are not described in detail.

The medical device 400 also has a body portion 410 with a recessed distal end portion 411 and a head portion 310 with a protruding proximal end portion 321, similar to medical device 300. The medical device 400 differs from medical device 300 in that it further has a second electrical cutting element 413 in addition to the first electrical cutting element 324. The second electrical cutting element 413 is positioned on the body portion 410, either at the same position or directly adjacent to the cutting edge 412, and opposite the first electrical cutting element 324. The second electrical cutting element may also be in the form of an RF electrode or an electrical heating element and may be disposed circumferentially on the inside of the recessed distal end portion 411 of the head portion 410, for example, in the form of an circumferential band. The second electrical cutting element 224 may be connected to a source of electrical energy with a second connector element 414 which is positioned within the body portion 410 and may extend along the length of the body portion 410. The second electrical cutting element 413 may be made from the same materials as the electrical cutting element 112 of medical device 100 (shown in FIGS. 1A and 1B).

The method of using the medical device 400 to form a fistula is substantially the same as for medical device 100 described above with respect to FIGS. 3A to 3G. It differs only in that, during the fistula formation step (see FIG. 3D), both the first and second electrical cutting elements 324, 413 are activated, resulting in the tissue of the arterial and venous walls being cut from both sides, thus making the fistula formation process more effective. Furthermore, during the fistula formation process, a medical practitioner may push the medical device 300 distally to allow the cutting edge 412 positioned on the body portion 410 to better engage the tissue of the arterial and venous walls.

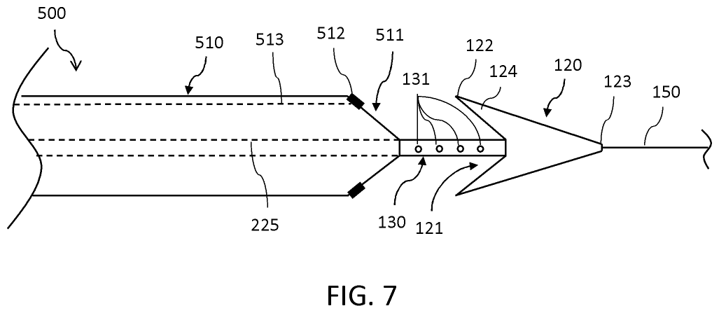

FIG. 7 shows another alternative embodiment of a medical device 500 for forming a fistula. The medical device 500 is similar to the medical device 100 of FIGS. 1 to 3, and the same reference numerals are used for identical features which are not described in detail here.

The medical device 500 also comprises a body portion 510 having a protruding distal end portion 511 with an electrical cutting element 512. However, the medical device 500 the differs from medical device 100 in that the electrical cutting element 512 is formed as a raised ridge, which is raised relative to the surface of the protruding distal end portion 511.

The electrical cutting element 512 being formed as a raised ridge increases the pressure being exerted on the tissue captured between the head portion 120 and the body portion 510. This may result in more effective cutting of both the electrical cutting element 512 and the cutting edge 122.

Various modifications will be apparent to those skilled in the art.

For example, the body portion 110, 310, 410, 510 may not be elongate or cylindrical but, may take any other suitable shape such as ellipsoidal or conical, for example.

The body portion 110, 310, 410, 510 may not have a protruding distal end portion or a recessed distal end portion. For example, the body portion 110, 310, 410, 510 may have a flat distal end portion.

The protruding distal end portion 111, 511 is not limited to being substantially conical or frustoconical but may take any other suitable shape. For example, the protruding distal end portion may be substantially hemispherical or pyramidal.

The recessed distal end portion 311, 411 is not limited to being a substantially conical or frustoconical cavity but may take any other suitable shape. For example, the recessed distal end portion may be a substantially hemispherical or pyramidal cavity.

The electrical cutting element 112, 324, 512 may be positioned anywhere on the distal end of the body portion or the proximal end of the head portion.

The electrical cutting element 112, 324, 512 is not limited to being a circumferential electrical cutting element but may take any suitable shape. For example, the electrical cutting element may comprise a number of individual portions positioned around the body portion or the head portion.

The second electrical cutting element 224, 413 may be positioned anywhere on the distal end of the body portion or the proximal end of the head portion. It is not limited to being positioned at the same position or adjacent to the cutting edge 122, 222, 312, 412.

The second electrical cutting element 224, 413 is also not limited to being a circumferential electrical cutting element but may take any suitable shape. For example, the second electrical cutting element 224, 413 may comprise a number of individual portions positioned around the body portion or the head portion.

The second electrical cutting element 224, 413 is not limited to being and RF electrode or an electrical heating element but may take any other suitable form.

The cutting edge 122, 222, 312, 412 may be positioned anywhere on the distal end of the body portion or the proximal end of the head portion.

The cutting edge 122, 222, 312, 412 is not limited to being a circumferential cutting edge but may be comprise a number of individual blades positioned around the distal end of the body portion or the proximal end of the head portion.

The cutting edge 122, 222, 312, 412 may comprise a serrated or a non-serrated blade.

The inner shaft 130 may not comprise any flushing ports 131 or a flushing lumen 133.

The head portion 120, 220, 320 may not have a substantially conical or tapered outer surface. For example, the outer surface of the head portion 120, 220, 320 may be substantially spherical, cylindrical, ellipsoidal, or pyramidal.

The head portion 120, 220, 320 may not have a recessed proximal end portion or a protruding proximal end portion. For example, the body portion 120, 220, 320 may have a flat distal end portion.

The recessed distal end portion 121, 221 is not limited to being a substantially conical or frustoconical cavity but may take any other suitable shape. For example, the recessed distal end portion may be a substantially hemispherical or pyramidal cavity.

The protruding proximal end portion 321 is not limited to being substantially conical or frustoconical but may take any other suitable shape. For example, the protruding proximal end portion may be substantially hemispherical or pyramidal.

The medical device 100, 200, 300, 400, 500 may not comprise a control handle 140.

The control handle 140 may not have capture control mechanism or a switch 142 for activating the electrical cutting element. The switch 142 may be positioned directly on the electrical power source, for example. In some embodiments, this electrical cutting element may not be activated with switch 142 but rather with a button, toggle, rotatable knob or a linear slider, for example.

The capture control mechanism is not limited to a linear slider 141 but may take any other suitable form. For example, the capture control mechanism may comprise a pull wire or a linear actuator activated with a switch.

The medical device 100, 200, 300, 400, 500 may not comprise a guidewire lumen 133.

All of the above are fully within the scope of the present disclosure and are considered to form the basis for alternative embodiments in which one or more combinations of the above-described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A medical device for forming a fistula, the medical device comprising:
a body portion having a longitudinal axis;
a head portion positioned distally of the body portion and configured to be longitudinally moveable relative to the body portion to capture tissue in which the fistula is to be formed between the head portion and the body portion;
a first electrical cutting element for cutting the tissue captured between the head portion and body portion; and
a cutting edge for mechanically cutting the tissue captured between the head portion and body portion.

2. The medical device of claim 1, wherein the medical device has a closed configuration where the head portion and the body portion are in contact with each other, and an open configuration where the head portion and the body portion are longitudinally spaced apart from each other.

3. The medical device of claim 2, wherein the first electrical cutting element and the cutting edge are disposed opposite each other on the body portion and head portion

4. (canceled)

5. The medical device of claim 1, wherein the first electrical cutting element and the cutting edge are disposed together on the head portion or the body portion.

6. The medical device of claim 5, wherein the first electrical cutting element and the cutting edge are disposed at the same position on the head portion or the body portion.

7. The medical device of claim 1, wherein the first electrical cutting element is circumferentially disposed on the head portion or the body portion.

8. The medical device of claim 1, wherein the cutting edge is a circumferential cutting edge.

9. The medical device of claim 1, wherein the first electrical cutting element is a resistive electrical heating element, or wherein the first electrical cutting element is an RF electrode.

10. (canceled)

11. The medical device of claim 1, wherein the body portion comprises a protruding distal end portion, and wherein the head portion comprises a complementary recessed proximal end portion for engaging the distal end portion of the body portion.

12. The medical device of claim 11, wherein the recessed proximal end portion of the head portion defines a cavity with a proximal opening.

13. The medical device of claim 11, wherein the cutting edge is positioned on a proximal leading edge of the recessed proximal end portion.

14. The medical device of claim 11, wherein the first electrical cutting element is positioned on the protruding distal end portion.

15. The medical device of claim 11, wherein the protruding distal end portion is substantially cone shaped.

16. The medical device of claim 15, wherein the first electrical cutting element is positioned at a base of the cone-shaped protruding distal end portion.

17. The medical device of claim 1, wherein the body portion comprises a recessed distal end portion, and wherein the head portion comprises a complementary protruding proximal end portion for engaging the distal end portion of the body portion.

18-22. (canceled)

23. The medical device of claim 1, further comprising a second electrical cutting element positioned opposite the first electrical cutting element on the body portion or the head portion.

24. The medical device of claim 23, wherein the second electrical cutting element is a resistive electrical heating element or an RF electrode.

25. The medical device of claim 23, wherein the second electrical cutting element is circumferentially disposed on the body portion or the head portion.

26-30. (canceled)

31. The medical device of claim 2, further comprising an inner shaft connected to the head portion and longitudinally moveable within the body portion, wherein the inner shaft comprises one or more flushing ports for injecting a contrast agent.

32-33. (canceled)

34. The medical device of claim 33, wherein, in the open configuration, the one or more flushing ports are disposed between the body portion and the head portion.

35-36. (canceled)
